(19) United States
(12) Patent Application Publication
(10) Pub. No.: US 2008/0044456 A1
(43) Pub. Date: Feb. 21, 2008

(54) Cellulose membrane drying process

(75) Inventor: Silvio Jose de Souza, Ibipora - Parana (BR)

Correspondence Address: MCCORMICK, PAULDING & HUBER LLP, CITY PLACE II, 185 ASYLUM STREET, HARTFORD, CT, 06103, US

(73) Assignee: Wellborn Participacoes Societarias S/C Ltda, Londrina (BR)

(21) Appl. No.: 11/505,603

(22) Filed: Aug. 17, 2006

(51) Int. Cl. A61K 31/717; A61K 9/70

(52) U.S. Cl. 424/443; 514/57

(57) ABSTRACT

A method of membrane drying of bacterial cellulose, characterized by the fact that the membrane is stuck to a device and under tension, for acquirement of a dry pellicle with plain, flat and flexible structure.

### SUMMARY OF THE INVENTION

The described material in the present invention is composed of a microbial membrane cellulose drought under tension, with aid of a leaked device, composed of inert and sanitary material that arrests the membrane only by the extremities and allows the exposition of the biomembrane in the two faces in direct contact with the flow of barren air, during the period of drying, getting as final product a dry pellicle with plain, flexible and flat structure.

An important aspect of the invention is that during the drying, the biomembrane keeps all characteristic of cleanness obtained in the chemical treatment, thus eliminating the risk of crossing contamination during the drying operation.

### TECHNICAL BACKGROUND

It is included in the state of the technique great amount of remedies and therapeutical methods to use the biocellulose as material to treat the injured skin.

The use of bacterial cellulose as compatible material is described in patents U.S. Pat. No. 4,912,049, BR P18404937 and BR PI 8602944, which demand its use as temporary substitute of skin.

This technology was diffused in several countries and the originality and newness of its properties are proven, as certify the works published for: Fontana et al. Acetobacter Cellulose Pellicle as a Temporary Skin Substitute; Applied Biochemistry and 5 Biotechnology, 1991, 28/25, 253-264; Gatti et Al. Physical Characterization of new biomaterial for wound management; Journal of Materials Science; Material in Medicine, 1994, 5, 190-193 that described that the material is permeable to gases and impermeable to liquids and Pitanguy I et al. Use of Cellulose Pellicle As Remedy; Brazilian magazine of Surgery, 10 1988; 78 (5) 317-326.

The production technologies are published in form of patents or in periodics about the subject, as example, we mention the work of Borzani & Souza “A simple method to control the bacterial production of cellulosic pellicles in order to obtain dried pellicles presenting a desired average thickness” in the Word Journal of Microbiology & Biotechnology, volume 14 of 1988, Fontana et al described “Acetobacter Cellulose Pellicle as a Temporary Skin Substitute in the Applied Biochemistry and Biotechnology vol 24/25 of 1990.

The biocellulose is produced by bacteria, specifically the Acetobacter xylinum, a gram-negative bacterium widely found in the nature and its main characteristic is to convert the glucose in form of fibrils and the interlacement at random of the fibrils results in a gelatinous pellicle.

The biocellulose membranes obtained of the fermentation contains impurities and pass through a chemical treatment to eliminate the non-cellulosics materials, as example the remaining of cells of ascetic bacteria and endotoxins that cause pyrogenics reactions in the organism. This purification is understood by the fact that the obtained membranes to be treated in one or more chemical solutions for elimination of the non-cellulosics materials, and preferential in a solution of SDS detergent of 0.1 to 3%, preferential 1%, followed by a solution of hydroxide of sodium of 0.5 to 5%, preferential 2%, followed by successive rinses for neutralization of the used products.

The U.S. Pat. No. 5,580,348 patent describes the use of hydroxide of sodium as form of purification of these biomembranes for medical use and Fontana et al. (1990, Appl. Biochem. Biotech, 24: 253-264), described that the bacterial cellulose used as temporary substitute of skin passes for treatment with a solution of SDS detergent at 1% followed by a strong alkali solution (hydroxide of sodium), thus eliminating the residual materials of the fermentation and converting the bacterial cellulose compatible to be used as primary remedy in the treatment of skin injuries.

The U.S. Pat. Nos. 5,580,518 and 5,722,646 patents describe that the membrane is placed in contact among absorbent materials to obtain its dehydration.

The U.S. Pat. No. 6,599,518 patent describes the use of organic solvent as methanol, ethanol, propyl alcohol, isopropanol, acetone and its mixtures for dehydration of the same ones.

The U.S. Pat. No. 5,846,213 patent describes that the biocellulose is dissolved in a system of solvent and is dried naturally on a support until the pellicle acquirement.

This invention describes the working of the system involving the drying by the forced circulation of filtered and warm air over the membrane, where it is fastened by the extremity of a leaked device remaining under tension during the drying process, guiding the staple fibers for a plain and flat final structure and allowing the exposition of the two faces directly with the flow of barren air, thus eliminating the risk of crossed contamination and maintaining the characteristic of cleanness obtained in the chemical treatment.

The invention is illustrated by the following examples:

### EXAMPLE 1

A membrane of biocellulose obtained through fermentation of a mean of culture in a not agitated environment by the Acetobacter xylinum bacterium, with dimensions of 48 cm×38 cm was rinsed, pressed and frozen. The pressing and freezing function is to remove the maximum amount possible of water from the membrane before submits it to the chemical treatment.

Afterwards, the biomembrane was placed soaking in a sodium sulphate lauril solution at 1% for 24 hours and rinsed until elimination of the sodium sulphate lauril excess and in sequence placed for more 24 hours soaking in a solution of hydroxide of sodium at 2%.

Followed by rinsing, until pH neutralization.

### EXAMPLE 2

A device constituted of two frames of picture in p.v.c was constructed, with external dimensions of 48 cm×38 cm and internal dimensions of 44 cm×34 cm, linked by a system of hinges in one lateral and a system of latch in the opposite lateral, where the humid pellicle gets stuck by the extremities in 2 cm. The leaked area of the device is of 1496 cm², where the stuck pellicle under tension only suffers the contact with the air flow.

### EXAMPLE 3

A pellicle was prepared according to example 1 and placed in the device according to example 2, and transferred to a greenhouse with air flow, until complete dehydration of the membrane.

The drying by air flow was performed in a greenhouse, that received the air insufflation through a machine whose system is based on the warm air circulation at 40° C. and filtered by a system of absolute filters type HEPA, combining this way, transference of heat (membrane heating) and of mass (humidity removal) with barren air.

The resultant material was the acquirement of a dry, fine, resistant, of plain structure, flexible and flat pellicle.

### EXAMPLE 4

The method of example 3 was repeated, except that it was performed test of electronic counting particle during the operation, it was used a calibrated particle accountant of the Climet brand, model CI500, series 989425.

The result found was Class ISO 7 (10.000).

The test was performed to certify that the installation, in the conditions as constructed and in operation, attend the class of cleanness for the manufacturing of health product.

### EXAMPLE 5

The method of example 3 was repeated, except that it was verified the temperature and relative humidity during the drying operation, it was used a calibrated controller of temperature and humidity brand Novus, model 321, serial number 0365307.

The temperature found in the beginning of the operation was of 24.9° C. (ambient temperature) and 65% of Relative Humidity and 40.0° C. and 28% of RH in the end of the operation. Then the temperature of the greenhouse was left to reach 25° C. and 65% RH to remove the device pellicle.

The test was performed to certify that the equipment that insufflates the warm air in the drying room attends to the delivery of heat to the membrane at the same time that extracts the humidity.

The resultant material was the acquirement of a dry, fine, resistant, of plain structure, flexible and flat pellicle.

### EXAMPLE 6

The method of example 3 was repeated, except that it was verified the temperature and relative humidity during the drying operation, it was used a calibrated controller of temperature and humidity brand Novus, model 321, serial number 0365307.

The temperature found in the beginning of the operation was of 24.9° C. (ambient temperature) and 65% of Relative Humidity and 79,8° C. and 16% of RH in the end of the operation. Then the temperature of the greenhouse was left to reach 25° C. and 65% RH to remove the device pellicle.

The test was performed to certify that the equipment that insufflates the warm air in the drying room attends to the delivery of heat to the membrane at the same time that extracts the humidity.

The resultant material was the acquirement of a dry, fine, resistant, of plain structure, flexible and flat pellicle.

### EXAMPLE 7

The method of example 3 was repeated, except that it was verified the temperature and relative humidity during the drying operation, it was used a calibrated controller of temperature and humidity brand Novus, model 321, serial number 0365307.

The temperature found in the beginning of the operation was of 24.9° C. (ambient temperature) and 65% of Relative Humidity and 25.1° C. and 64.7% of RH in the end of the operation.

The test was performed to certify that the equipment that insufflates the warm air in the drying room attends to the delivery of heat to the membrane at the same time that extracts the humidity.

The resultant material was the acquirement of a dry, fine, resistant, of plain structure, flexible and flat pellicle.

### EXAMPLE 8

The method of example 3 was repeated, except that the resultant dry pellicle of the drying process was sealed up in a “pouch” and sent for evaluation of the microbial load (Bioburden). The way used for the assay was the Trypitic Soy Agar and Sabouraud Dextrose Agar, with incubation of 48 hours at 37° C. for bacteria and 5 days at 28° C. for fungus.

As result it was found 0 CFU/unity in the counting of bacteria and 0 CFU/unity in the counting of fungus. The test was performed to certify that the operations and the contact of the membrane with the air flow attend to the class of cleanness for the production of health product.

*CFU—Colony Former Unity

1- A method of membrane drying of bacterial cellulose, characterized by the fact that the membrane is stuck to a device and under tension, for acquirement of a dry pellicle with plain, flat and flexible structure.

2- A method of drying according to item 1, characterized by the fact that the drying occurs by circulation of a barren air flow.

3- A method of drying according to item 2, characterized by the fact that the air flow is barren by a system of absolute filter type HEPA.

4- A method of drying according to items 1 to 3, characterized by the fact that the membrane of microbial cellulose is stuck by the extremities to a device during the drying process.

5- A method of drying according to item 4, characterized for the fact that the device is leaked in the central part, constituted of two frames of sanitary material stuck between themselves, by a system of hinges, that ties the membrane of cellulose by the extremities, in all the frame extension, keeping it under tension.

6- A method of drying according to item 5, characterized by the fact that the material of the device is preferential of p.v.c or similar material, inert and with flat and washable surface.

7- A method of drying according to items 1 to 6, characterized by the fact that the membrane is under tension during the process of drying for acquirement of a plain and flat structure pellicle.

8- A method of membranes drying of microbial cellulose for use in the medical area, characterized for including the stages of:
a—Packaging of the membrane of microbial cellulose purified in a device for drying under tension in a barren air flow;
b—The air flow is warm preferential in a resistance system until the temperature of 80 centigrade degrees;
c—The used temperature preferential is of 40 centigrade degrees;
d—This device is suspended in a support, in the vertical or horizontal position, in such way that the two faces of the microbial cellulose membrane are exposed to the air flow;
e—The drying process allows that the membrane dries in the strained and plain form and without contact with another type of material.

9- The process according to the claim 1, characterized by the fact that the microbial cellulose is produced by the Acetobacter xylinun microorganism.

10- The process according to the claim 1, characterized by the fact that it uses a chemical treatment for purification of the membranes, making them biocompatible.

11- A process according to the claim 1, characterized by the fact that associate in the chemical treatment for the purification the hydroxide of sodium and lauril sodium sulphate products.

12- A process according to the claim 4, using the chemical products lauril sodium sulphate and hydroxide of sodium in the concentration of 0.5 to 2.0% in p/v.
